(19) United States
(12) Patent Application Publication
(10) Pub. No.: US 2026/0363723 A1
(43) Pub. Date: Nov. 19, 2026

(54) Surgical Instrument

(71) Applicant: Pajunk GmbH Medizintechnologie, Geisingen (DE)

(72) Inventors: Simone PAJUNK-SCHELLING, Geisingen (DE); Martin HAUGER, Donaueschingen (DE)

(73) Assignee: Pajunk GmbH Medizintechnologie, Geisingen (DE)

(21) Appl. No.: 19/515,779

(22) PCT Filed: Aug. 29, 2024
(86) PCT No.: PCT/EP2024/074127

§ 371 Date: Mar. 2, 2026

(30) Foreign Application Priority Data
Aug. 30, 2023 (DE) 1020231233408

(51) Int. Cl. A61B 17/32; A61B 17/00; A61B 17/34

(52) U.S. Cl. CPC A61B 17/320036; A61B 17/3403; A61B 2017/00473; A61B 2017/00526; A61B 2017/3413

(57) ABSTRACT

A surgical instrument having a handle and a blade holder disposed thereon with a blade disposed in a plane (EK), where the blade comprises a concavely developed cutting edge, and where the blade holder comprises a width (bK) in the direction transverse to a longitudinal axis (lK) of the blade holder. Additionally, the blade holder comprises an upper side and a lower side, such that the plane extends transverse to the upper side and to the lower side. The upper side comprises at least one recess with a side wall, a bottom surface, a length (lA) in the direction of the longitudinal axis (lK) of the blade holder and a width (bA) in the direction transverse to the plane (EK) of the blade, such that the width (bA) of the recess is greater than 75% of the width (bK) of the blade holder.

### CROSS-REFERENCE TO RELATED APPLICATIONS

This application is a U.S. national stage application under 35 U.S.C. § 371 of International Application No. PCT/EP2024/074127, filed Aug. 29, 2024, which claims priority to German Patent Application No. 10 2023 123 340.8, filed Aug. 30, 2023.

### FIELD OF DISCLOSURE

The present disclosure relates to a surgical instrument.

### BACKGROUND

The median nerve of the hand that supplies the muscles of the thumb and the metacarpus runs through a tunnel-like tube, the so-called carpal tunnel. At the level of the wrist a broad ligament stretches between the musculature of the thumb and that of the little-finger. This ligament, also referred to as retinaculum flexorum, is the roof of the carpal tunnel which includes the median nerve and the flexor tendons of the fingers. In carpal tunnel syndrome this channel becomes too narrow whereby pressure onto the median nerve is generated. This can lead to complaints, for example in the form of tingling or numbing of the hands.

In some cases treatment of carpal tunnel syndrome requires surgical intervention. In the surgical procedure the nerve channel is expanded thereby that the retinaculum flexorum, which spans over the carpal bones and delimits the carpal tunnel toward the palm, is severed. This intervention can be carried out as an open procedure as well as also as a minimally invasive procedure. In the case of a feasible minimally invasive procedure for carpal tunnel roof splitting, an approximately 2 cm long incision is placed in the region of the flexor aspect of the wrist, through which a small probe with a small scalpel is introduced into the carpal tunnel between the median nerve and the carpal tunnel roof. When the probe is located in the carpal tunnel the carpal tunnel roof can be split from the inside.

For minimally invasive procedures is known a surgical instrument with a handle and a blade with a concavely developed cutting edge disposed thereon, wherein the blade comprises a plane. For example, DE 10 2019 120 671 A1 discloses such surgical instrument which comprises ultrasound visibility-enhancing means in the form of a multiplicity of indentations which comprise in each case precisely three side faces oriented perpendicularly to one another. Even if such indentations enable a certain ultrasound visibility, it becomes apparent, however, that the ultrasound visibility in this instrument is in need of improvement.

### SUMMARY

Therefore the problem addressed by the disclosure described herein comprises providing a surgical instrument with which the safety of the minimally invasive intervention on the carpal tunnel of a hand can be further enhanced.

The surgical instrument, according to embodiments of the present disclosure, have a handle and a blade holder disposed thereon with a blade disposed in a plane, such that the blade comprises a concavely developed cutting edge, wherein the blade holder has a width in the direction transversely to a longitudinal axis of the blade holder, is distinguished thereby that the blade holder has an upper side and a lower side. As such, the plane extends transversely, in particular perpendicularly, to the upper side and to the lower side, and where the upper side comprises at least one recess with a side wall, a bottom surface, a length in the direction of the longitudinal axis of the blade holder and a width in the direction transversely to the plane of the blade. Moreover, according to these embodiments of the present disclosure, the width of the recess is greater than 75%. In some embodiments, the width of the recess is greater than 80%, in particular greater than 85% of the width of the blade holder. It has surprisingly been found that a recess of such size is especially clearly visible in ultrasound frequencies and therewith can significantly enhance the ultrasound visibility.

The width of the recess is herein in particular less than the width of the blade holder.

The bottom surface is advantageously oriented perpendicularly to the plane of the blade. Such disposition can enable good localization of the blade since the relative orientation between bottom surface and blade is fixed.

According to embodiments of the present disclosure, the bottom surface is disposed parallel to the upper side. Since in the specific operation for treatment of carpal tunnel syndrome an ultrasound transducer, as a rule, is brought perpendicularly onto the upper side of the blade holder, such disposition can further enhance the ultrasound visibility.

In embodiments of the present disclosure, the side wall, in particular in all four side directions, is disposed perpendicularly to the upper side. Since in the specific operation for the treatment of carpal tunnel syndrome the ultrasound as a rule is brought perpendicularly onto the upper side of the blade holder, such disposition can further improve the ultrasound visibility.

In embodiments of the present disclosure, the length of the recess is greater than the width of the recess. The physical form of the recess resulting therewith can enable good visibility in the ultrasound image.

Embodiments of the present disclosure provide that the at least one recess comprises parallel to the upper side a rectangular cross section with a front edge, a rearward edge and two side edges. Such recess can enable an especially good visibility in the ultrasound image.

According to embodiments of the present disclosure, one of the at least one recesses is disposed such that the front edge is disposed in the same sectional plane perpendicularly to the longitudinal axis of the blade holder as is the cutting edge, in particular the base of the concave cutting edge. Stated differently, the base or the lowest point of the cutting edge in top view lies underneath the front edge of the recess. Such physical form can enable especially good localization of the blade since the relative orientation between recess and blade is fixed.

The ultrasound visibility can be further improved if, according to embodiments,, several recesses, such as three recesses, are disposed in the upper side of the blade holder sequentially in the direction of the longitudinal axis of the blade holder, since such structure can also generate a clearly visible structure in the ultrasound image.

In embodiments, the front edges of the recesses are disposed equidistantly with respect to one another. With the aid of the recesses a type of ruler can be formed in this way which can provide the user with information regarding the penetration depth of the instrument or the distances to components of the hand surrounding the instrument.

In embodiments, as a rule, the ultrasound visibility of the recess is better the deeper the recess is. Therefore, the side wall has as high a height as is feasible, for example a height amounting to up to 50% of the blade holder.

In embodiments, to improve the ultrasound visibility still further, in the bottom surface several ultrasound reflectors are disposed which are developed in particular as indentations, wherein the indentations comprise in each instance two or three side walls each oriented perpendicularly with respect to one another.

In embodiments, the blade is fabricated of metal, in particular of high-grade steel. A blade of metal in the desired sharpness can be fabricated especially well.

In embodiments, the blade holder and/or the handle are fabricated of synthetic material, particularly in injection molding processes or 3-D printing. A simple and cost-effective production process can thereby be enabled. Moreover, the surgical instrument can in this way be produced with a lower weight than, for example, in the fabrication of metal.

According to embodiments of the present disclosure, the blade is nondetachably disposed in the blade holder, in particular be disposed therein such that it is overmolded into it in an injection molding process or encased using a 3D printing process. In this way reliable fixing of the blade in the surgical instrument can be attained.

An additional aspect of the present disclosure provides for the blade holder to be detachably connected with the handle. In this manner there is the feasibility of developing the blade holder as a disposable component, however of utilizing the handle again multiple times.

In embodiments, the handle comprises through-holes by which the weight of the handle can be reduced.

### BRIEF DESCRIPTION OF THE DRAWINGS

An embodiment example of the present disclosure will be explained in detail in conjunction with the following Figures. Therein depict:

FIG. 1 is a perspective view of an embodiment example of a surgical instrument, according to embodiments of the present disclosure.

FIG. 2 is a detail enlargement of the distal end of the surgical instrument of FIG. 1, according to embodiments of the present disclosure.

FIG. 3 is a top view onto the surgical instrument of FIG. 1, according to embodiments of the present disclosure.

FIG. 4 is a section along line A-A of FIG. 3, according to embodiments of the present disclosure.

FIG. 5 is a detail enlargement of FIG. 4, according to embodiments of the present disclosure.

FIG. 6 is a detail enlargement of FIG. 2, according to embodiments of the present disclosure.

### DETAILED DESCRIPTION

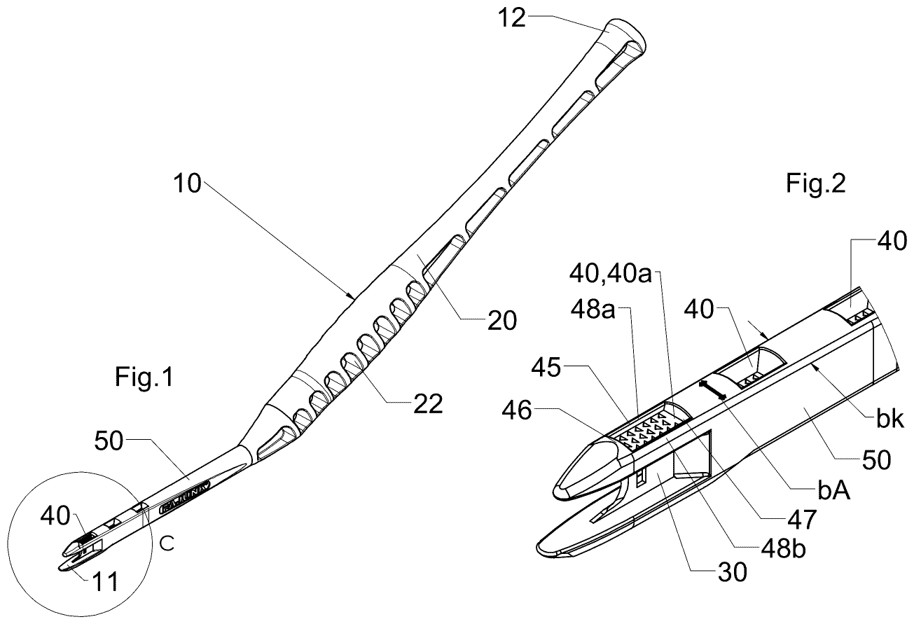

FIGS. 1 to 6 show different views of an embodiment example of a surgical instrument 10 according to embodiments of the present disclosure.

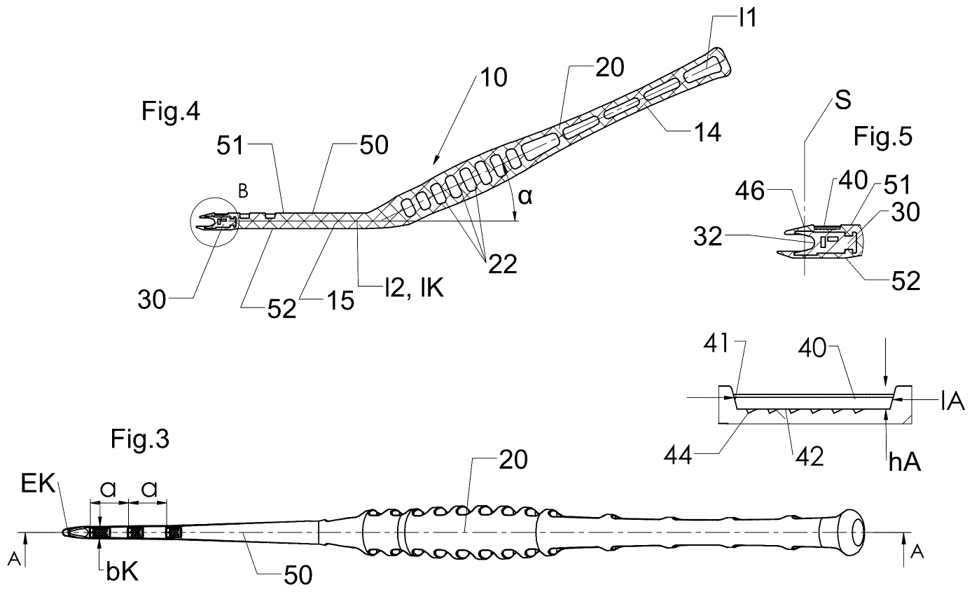

The instrument 10 comprises a handle 20 and a blade holder 50 disposed thereon. The blade 30 comprises a concavely developed cutting edge 32. As is discernible in particular in FIG. 5, the blade 30 can be developed foliately and thereby comprises a plane EK which extends parallel to a surface. In FIG. 5 the plane EK of blade 30 is the plane of the paper and in FIG. 3 extends perpendicularly to the paper plane. The thickness of the foliate blade 30 should be sufficient to ensure adequate stability. The blade 30 is in particular fabricated of metal, for example of high-grade steel. The cutting edge 32 can, for example, be developed in U form or V form. The cutting edge 32 lies in particular in the plane EK of the blade 30. For example starting from one of the side edges of the foliate blade 30, the cutting edge 32 can be developed as a concave recess.

Blade 30 is secured in the blade holder 50 which is disposed on the handle 20. The blade holder 50 can herein be connected unitarily with the handle 20 or be disposed detachably on the handle 20 across a detachable connection, for example a screw connection or a bayonet connection. This enables the simple exchange of the blade 30 thereby that the blade 30 including the blade holder 50 can be exchanged.

The instrument 10 can comprise a first segment 14, formed largely for example by the handle 20, with a first longitudinal axis 11 and a second segment 15, formed for example largely by the blade holder 50, with a second longitudinal axis 12. The longitudinal axes 11 and 12 can be aligned. However, the second longitudinal axis 12 is advantageously disposed at an angle a with respect to the first longitudinal axis 11, wherein the angle α is in the range of 10° to 40°, in the range of 20° to 30° and, for example, in some embodiments, is 25°. Herein the angle a, by which the second longitudinal axis 12 is set with respect to the first longitudinal axis 11, is in particular disposed in the plane EK of blade 30, as shown in FIG. 4.

The instrument 10 can be developed in the form of a pen with a distal end 11 and a proximal end 12, wherein the blade 30 is disposed on the distal end 11, in particular frontally on the distal end 11. The blade 30 is herein oriented in particular such that the longitudinal axis 12 of the first segment 14, in particular of the handle 20, lies in the plane of the blade 30.

The blade holder 50 comprises a longitudinal axis IK which, in particular, can coincide with the second longitudinal axis 12, a width bK as well as an upper side 51 and a lower side 52, wherein the plane EK of the blade 30 extends transversely, in particular perpendicularly, to the upper side 51 and to the lower side 52. The upper side 51 comprises at least one recess 40 with a side wall 41, a bottom surface 42, a length lA in the direction of the longitudinal axis lK of the blade holder 50 and a width bA in the direction transversely to the plane EK of blade 30. The width bA of recess 40 is greater than 75%, greater than 80%, or greater than 85% of the width bK of the blade holder. The recess 40 extends in particular nearly over the entire width bK of the blade holder 50 insofar that the side wall 41 of the recess 40 can still be developed stably in the blade holder 50. However, therewith the width bA of the recess 40 is less than the width bK of the blade holder 50. The length lA of recess 40 is preferably greater than the width bA of recess 40. The bottom surface 42 can be oriented perpendicularly to the plane EK of blade 30. The bottom surface 42 can be disposed in particular parallel to the upper side 51. The side wall 51 can be disposed perpendicularly to the upper side 51.

The at least one recess 40 is developed in particular such that, parallel to the upper side 51 it comprises a rectangular cross section with a front edge 46, a rearward edge 47 and two side edges 48a, 48b oriented in particular parallel with respect to one another. The recess 40 can in particular be developed substantially in the form of a cuboid.

The embodiment example of the surgical instrument 10 depicted in the Figures comprises three recesses 40 which are disposed in the upper side 51 of the blade holder 50 sequentially in the direction of the longitudinal axis lK of the blade holder 50. Recess 40a which is closest to the distal end 11 of the instrument 10 is in particular disposed such that the front edge 46 is disposed above the blade 30, in particular above the most proximal point of the cutting edge 32 or, stated differently, in the same sectional plane S perpendicularly to the longitudinal axis lK of the blade holder 50 as is the cutting edge 32, in particular the base of the concave cutting edge 32 or the most proximal end of the cutting edge 32, as shown in FIG. 5.

The three recesses 40 are in particular disposed such that the front edges 46 of recesses 40 are disposed equidistantly at a spacing a with respect to one another, as shown in FIG. 3, whereby a type of ruler can be formed. The spacing a can be, for example, 1 cm.

The side wall 41 of recess can have a height hA which amounts to up to 50% of the height hK (not shown in the Figures) of the blade holder 50. The recess 40a which is disposed closest to the distal end 11 of instrument 10 and in particular is disposed above the blade 30, can herein have a lesser depth than the following recesses 40 since this recess 40a is located above the blade 30.

In the bottom surface 42 ultrasound reflectors can be disposed which can be developed as indentations 44. The indentations 44 are in particular developed such that they reflect ultrasound frequency, in particular independently of the direction from which the ultrasound impinges on the indentations 44. For this purpose, the indentations 44 can comprise for example precisely three side faces 44a, 44b, 44c oriented perpendicularly with respect to one another, as shown in FIG. 2. Thereby an indentation 44 results such as if a corner of a cube had been pressed into a surface. Such indentations 44 reflect back an incident ultrasound beam, independently of the direction of the incidence, parallel to the direction of incidence.

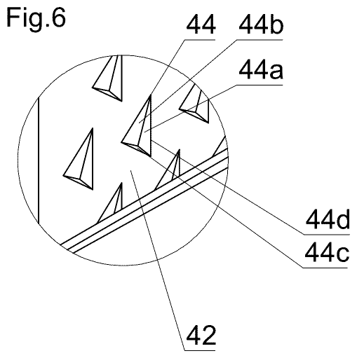

A first side face 44a of the indentation 44 can form with the bottom surface 42 an angle of approximately 35°. A second side face 44 b and a third side face 44 c can be disposed symmetrically with respect to a longitudinal axis lK of the blade holder IK 50. For example, the indentation 44 can have a triangular base face 44d in the bottom surface 42, wherein the tip of the triangle points to the distal end 11 of the surgical instrument 10, as shown in FIGS. 2 and 6.

The blade holder 50 and/or the handle 20 can be fabricated of a synthetic material, for example employing an injection molding process or a 3D printing process, wherein in particular the blade holder 50 and the handle 20 can be developed as two parts or also connected integrally as a single part. In particular the blade 30 can herein be disposed nondetachably in the blade holder 50, for example thereby that the blade 30 is directly overmolded in the injection molding process or is directly encased in the 3D printing process. The handle 20 can comprise through-holes 22 by means of which the weight of the 20 can be reduced.

### LIST OF REFERENCE NUMBERS

10 Instrument
11 Distal end
12 Proximal end
14 First segment
15 Second segment
20 Handle
22 Through-hole
30 Blade
32 Cutting edge
40 Recess
41 Side wall
42 Bottom surface
44 Indentation
44a First side face
44b Second side face
44c Third side face
44d Base face
45 Cross section
46 Front edge
47 Rearward edge
48a Side edge
48b Side edge
50 Blade holder
51 Upper side
52 Lower side
EK Plane of the blade
lK Longitudinal axis of the blade holder
l1 First longitudinal axis
l2 Second longitudinal axis α Angle
lA Length of recess
bA Width of recess
hA Height of recess
bK Width of blade holder
hK Height of blade holder
S Sectional plane
a Distance

1. A surgical instrument comprising:
a handle and a blade holder disposed thereon with a blade disposed in a plane (EK); wherein the blade comprises a concavely developed cutting edge;
wherein the blade holder comprises a width (bK) in the direction transverse to a longitudinal axis (IK) of the blade holder;
wherein the blade holder comprises an upper side and a lower side;
wherein the plane (EK) extends transverse to the upper side and to the lower side, such that the upper side comprises at least one ultrasound visibility-enhancing recess with a side wall, a bottom surface, a length (lA) in the direction of the longitudinal axis (lK) of the blade holder and a width (bA) in the direction transverse to the plane (EK) of the blade; and
wherein the width (bA) of the recess is greater than 75% of the width (bK) of the blade holder

2. The surgical instrument of claim 1, wherein the width (bA) of the recess is less that the width (bK) of the blade holder.

3. The surgical instrument of claim 2, wherein the bottom surface is oriented perpendicularly to the plane (EK) of the blade.

4. The surgical instrument of claim 3, wherein the bottom surface is disposed parallel to the upper side.

5. The surgical instrument of claim 4, wherein the side wall is disposed perpendicularly to the upper side.

6. The surgical instrument of claim 5, wherein the length (lA) of the recess is greater than the width (bA) of the recess.

7. The surgical instrument of claim 6, wherein the at least one recess parallel to the upper side comprises a rectangular cross section with a front edge, a rearward edge and two side edges.

8. The surgical instrument of claim 7, wherein one of the at least one recesses is disposed such that the front edge is disposed in the same sectional plane(S) perpendicular to the longitudinal axis (lK) of the blade holder as the cutting edge, in particular the base of the concave cutting edge.

9. The surgical instrument of claim 8, wherein several recesses are disposed in the upper side of the blade holder sequentially in the direction of the longitudinal axis (lK) of the blade holder.

10. The surgical instrument of claim 9, wherein the front edges of the recesses are disposed equidistantly with respect to one another.

11. The surgical instrument of claim 10, wherein the side wall comprises a height (hA) which amounts to up to 50% of the height (hK) of the blade holder.

12. The surgical instrument of claim 11, wherein in the bottom surface several ultrasound reflectors are disposed which are in particular developed as indentations, wherein the indentations especially preferably comprise each two, preferably precisely three, side walls oriented in each instance perpendicularly with respect to one another.

13. The surgical instrument of claim 12, wherein the blade is fabricated of metal, in particular of high-grade steel.

14. The surgical instrument of claim 13, wherein the blade holder and/or the handle are fabricated of synthetic material, in particular in an injection molding process or by 3D-printing.

15. The surgical instrument of claim 14, wherein the blade is disposed nondetachably in the blade holder, in particular is injection molded therein in an injection molding process or is disposed jacketed in 3D printing.

16. The surgical instrument of claim 15, wherein the blade holder is detachably connected with the handle.

17. The surgical instrument of claim 16, wherein the handle comprises through-holes.
